(19) United States
(12) Patent Application Publication
(10) Pub. No.: US 2026/0363843 A1
(43) Pub. Date: Nov. 19, 2026

(54) BIOLOGIC PELVIC SUSPENSION METHOD USING CADAVERIC TISSUE CONSTRUCTS FOR PELVIC SUPPORT

(71) Applicant: ARMS Inc., Fort Lauderdale, FL (US)

(72) Inventors: Robert Greer, Fort Lauderdale, FL (US); Ty Bolton Erickson, Henderson, NV (US)

(21) Appl. No.: 19/668,097

(22) Filed: May 5, 2026

Related U.S. Application Data
(60) Provisional application No. 63/804,999, filed on May 13, 2025.

(51) Int. Cl. A61F 2/00

(52) U.S. Cl. CPC A61F 2/0063

(57) ABSTRACT

This document describes a method for performing a surgical procedure to treat a pelvic organ prolapse of a patient. The method comprising attaching one or more fibrous connective tissue samples to a vaginal wall of the patient comprising a posterior vaginal wall and an anterior vaginal wall. Attaching the one or more fibrous connective tissue samples comprises: attaching a first portion of the one or more fibrous connective tissue samples to the posterior vaginal wall; and attaching a second portion of the one or more fibrous connective tissue samples the anterior vaginal wall. The method also includes attaching a third portion of the one or more fibrous connective tissue samples to a ligamentous structure at a sacral promontory of the patient such that the one or more fibrous connective tissue samples apply a tension to the vaginal wall to treat the pelvic organ prolapse of the patient.

### CROSS REFERENCE TO THE RELATED APPLICATION

This application claims the benefit of U.S. Provisional Application No. 63/804,999, filed May 13, 2025, the contents of which are incorporated by reference herein.

### TECHNICAL FIELD

This document relates to systems and methods for performing pelvic suspension and support procedures such as sacrocolpopexy, for example by using a human tissue sample to provide structural support to one or more pelvic organs.

### BACKGROUND

Sacrocolpopexy is a surgical procedure performed to correct pelvic organ prolapse. One form of pelvic organ prolapse is vaginal vault prolapse, a condition in which the upper portion of the vagina loses support and descends, sometimes following hysterectomy. In certain sacrocolpopexy procedures, a mesh implant is used to attach the vaginal vault to the sacrum, a triangular bone located at the base of the spine. This attachment repositions the vagina toward a pre-prolapse anatomical location and is intended to provide long-term pelvic support. Such procedures may be performed using open, laparoscopic, or robotic-assisted surgical techniques.

In some cases, sacrocolpopexy is performed to address symptoms associated with pelvic organ prolapse, which may include sensations of vaginal pressure or bulging, difficulties with urination or bowel movements, and discomfort during sexual activity. Sacrocolpopexy can be suitable as a pelvic suspension procedure depending on factors such as patient anatomy, prior surgical history, and the desired durability of pelvic support. In some cases, sacrocolpopexy can be used for patients who have experienced prior pelvic repair procedures or who are seeking a longer-term pelvic support option. Sacrocolpopexy can be a popular procedure because of its high long-term success rate and lower risk of prolapse recurrence as compared with other procedures.

### SUMMARY

This document describes devices, systems, and methods for performing a pelvic suspension procedure using tissue to provide structural support to pelvic anatomy. In some embodiments, the pelvic suspension procedure is a sacrocolpopexy used to treat pelvic organ prolapse of a patient. For example, vaginal vault prolapse is a condition in which the upper portion of the vaginal canal loses support and descends. Sacrocolpopexy is one pelvic suspension procedure that addresses this condition by restoring support to the upper portion of the vaginal canal and repositioning the canal toward a pre-prolapse anatomical location. In some embodiments, a sacrocolpopexy involves securing one or more fibrous connective tissue samples to pelvic anatomy, including attachment to tissue associated with the vaginal wall and fixation to a structure at or near the sacral promontory. The sacral promontory is part of the spinal anatomy and may provide a stable anchoring location for supporting the fibrous connective tissue samples. In some embodiments, the techniques described herein can be applied to support different pelvic anatomical structures using a common fixation framework, including attachment to soft tissue structures and fixation to a ligamentous structure proximate to the sacrum. Such techniques can be used in connection with procedures involving the vaginal vault, uterus or cervix, or colorectal structures.

In some cases, the one or more fibrous connective tissue samples include tendon-based tissue, such as tissue derived from a tibialis tendon. Such tissue may be obtained from donor sources and processed to reduce or remove cellular components while preserving structural features of the extracellular matrix (ECM). Processing of the tissue may reduce the likelihood of an adverse host response following implantation. Such adverse host responses can include immune responses, and rejection of the implant.

The ECM of tendon-based tissue can include a collagen-rich fibrous architecture that provides directional load-bearing characteristics. In certain embodiments, the collagen fibers may be substantially aligned along a primary axis, which can facilitate transmission of tensile forces when the construct is placed under load. These structural characteristics can make tendon-based biologic constructs suitable for use in pelvic suspension and support procedures. Tendon-based tissue can differ structurally from other biologic materials such as allograft dermis and xenograft tissues, becasue tendon-based tissue exhibits a dense, anisotropic collagen architecture adapted for directional load bearing. Characteristics of tendon-based tissue such as geometry, orientation, and fixation can provide benefits when the tissue is used as a load-bearing support. Directional mechanical properties of tendon-based tissue, and the ability of tendon-based tissue to sustain tensile loads over extended spans between fixation points are important mechanical characteristics.

Sacrocolpopexy is a pelvic suspension procedure that may be used to address apical pelvic organ prolapse by attaching the vaginal vault to anatomy at or near the sacral promontory. In some embodiments, the procedure involves securing a posterior portion of a support construct to tissue associated with a posterior region of the vaginal wall, securing an anterior portion of the support construct to tissue associated with an anterior region of the vaginal wall, and securing another portion of the support construct to a fixation site at or near the sacral promontory. In some embodiments, the support construct comprises a tendon-based biologic tissue. Tendon-based tissue can exhibit a fibrous collagen architecture adapted for directional load transmission and may be configured to transfer tensile forces between fixation sites when placed under load. These characteristics can make tendon-based biologic constructs suitable for use in pelvic suspension and support procedures.

In one embodiment, the support construct comprises a single tendon-based biologic tissue sample that is folded over itself to attach to both the posterior and anterior vaginal walls, the folded over portion attaching to the sacral promontory. In another embodiment, the support construct comprises two separate tendon-based biologic tissue samples, one sample attaching to the posterior vaginal wall and the sacral promontory and another sample attaching to the anterior vaginal wall and the sacral promontory. In both examples, the tendon-based material maintains the pelvic organ in place to provide support that has been lost because of one or more patient conditions or related procedures (e.g., hysterectomy).

In some embodiments, the support construct comprises a single tendon-based biologic tissue configured as a bifurcated construct, such as a Y-shaped configuration, having multiple portions adapted for attachment to different regions of pelvic anatomy. For example, the construct may include a first portion configured for attachment to a posterior pelvic support site, a second portion configured for attachment to an anterior pelvic support site, and a sacral portion configured for fixation at or near the sacral promontory. In some embodiments, the bifurcated construct is formed from a single elongate tendon, thereby providing a continuous load path between the attachment portions when the construct is placed under tension.

In some cases, pelvic suspension procedures such as sacrocolpopexy can be performed through a minimally invasive laparoscopic or robotic approach. For example, a clinician can use small incisions in the abdomen to insert a laparoscope or robotic instruments, which provide enhanced visualization and precision. Tendon-based biologic tissue samples can be attached to the vaginal cuff (e.g., the posterior and anterior walls) and secured to a fixation site at or adjacent the sacral promontory to restore normal vaginal anatomy and support. Robotic assistance can offer improved dexterity and three-dimensional (3D) visualization, which can facilitate suturing in deep pelvic spaces. This approach can result in lower pain and discomfort levels, shorter hospital stays, and faster recovery as compared with open surgery methods.

Synthetic mesh can be used in sacrocolpopexy procedures to provide mechanical durability and structural support under load. However, in some patients, synthetic mesh can introduce several complications including tissue erosion, mesh exposure, inflammatory response, infection, and migration. These complications can lead to safety issues or patient reluctance. While sacrocolpopexy using mesh remains a preferred approach for many surgeons, a biologic alternative that preserves the mechanical advantages of sacrocolpopexy—while addressing patient safety and preference—provides a clinical advancement.

In particular examples described herein, a method for performing a surgical procedure to treat a pelvic organ prolapse of a patient comprises attaching one or more fibrous connective tissue samples to a vaginal wall of the patient comprising a posterior vaginal wall and an anterior vaginal wall. Attaching the one or more fibrous connective tissue samples comprises: attaching a first portion of the one or more fibrous connective tissue samples to the posterior vaginal wall and attaching a second portion of the one or more fibrous connective tissue samples to the anterior vaginal wall. The method also includes attaching a third portion of the one or more fibrous connective tissue samples to a ligamentous structure proximate to the sacral promontory of the patient such that the one or more fibrous connective tissue samples apply a tension to the vaginal wall to treat the pelvic organ prolapse of the patient.

Particular embodiments of the subject matter described in this document can be implemented to realize one or more of the following advantages. First, using human tissue to secure a position of the vaginal canal as opposed to using synthetic materials (e.g., a polymer mesh) or using non-human tissue materials can be beneficial because human tissue can attach to the patient’s vaginal wall, which may reduce the likelihood of migration across one or more physiological membranes. In some embodiments, biologic tissue derived from human fibrous connective tissue can exhibit compatibility with surrounding pelvic tissue and may be configured for integration with host tissue when implanted. If an implanted material is synthetic material or non-human tissue material, because such a material is foreign to human tissue, migration can occur when human tissue grows or moves around the implanted material. Synthetic material can also cut into human tissue over time. Using one or more fibrous connective tissue samples to secure the position of the vaginal canal can provide a similar result as compared with mesh procedures while avoiding some complications that arise with mesh procedures over time. For example, the fibrous connective tissue samples can provide similar strength, durability, and longevity as compared with synthetic or non-human materials, the biologic properties of the fibrous connective tissue may reduce the likelihood of the tissue from migrating across one or more physiological membranes (e.g., bladder, vaginal wall, rectum). In some embodiments, fibrous connective tissue used in the support construct includes an organized collagen architecture capable of transmitting tensile loads between fixation sites. When configured as described herein, the support construct may be suitable for use in pelvic suspension procedures as an alternative to permanent synthetic materials in selected clinical contexts.

Second, in embodiments where the tendon-based biologic tissue comprises a tibialis tendon, the biologic tissue includes a dense, longitudinally organized collagen architecture comprising Type I collagen fibers, which is adapted to transmit tensile loads along its length. Fibrous connective tissue such as the tibialis tendon can represent strong tissue that connects muscle to bone. Because the tibialis tendon includes Type I collagen fibers that are highly organized to handle tensile loads, the tibialis tendon can be particularly suited for securing a position of the vaginal canal relative to the sacral promontory. Moreover, the tibialis tendon can be subjected to the force of the tibialis anterior muscle located in the leg, which is subjected to high amounts of force throughout the life of a human. As a result, fibrous connective tissue of the tibialis tissue can provide sufficient strength and durability for maintaining a pelvic organ in position for long periods of time. Furthermore, the structural characteristics of tibialis tendon tissue can make it suitable for use in pelvic suspension constructs configured to transfer tensile forces between pelvic support sites and a sacral fixation site. In some embodiments, the tibialis tendon is selected based on its native geometry and fibrous organization, which may facilitate formation of a bifurcated support construct.

Third, using human tissue can result in increased comfort for the patient as compared with solutions involving synthetic materials. For example, when synthetic materials migrate over time, this can cause synthetic materials to encroach through membranes or into spaces where the synthetic materials were not intended to occupy. In the case of a synthetic mesh sacrocolpopexy procedure, this can involve the synthetic mesh crossing the vaginal wall into the vagina, entering the bladder wall, entering the rectal wall, or migrating into other areas of tissue. This encroachment can lead to pain and discomfort. Using human tissue instead of a synthetic material may prevent such pain and discomfort by reducing the likelihood of migration into unwanted areas.

Fourth, the use of biologic tissue in a pelvic suspension construct can influence an interaction between the implant and surrounding pelvic tissue. Biologic tissues derived from human fibrous connective tissue can exhibit mechanical compliance and surface characteristics that differ from permanent synthetic materials. These material characteristics can make biologic tissue an ideal support construct for use in pelvic suspension procedures, such as in contexts where long-term implantation of synthetic materials is undesirable.

Fifth, there may be a decreased likelihood that the body will reject human tissue as compared with synthetic or non-human material. Synthetic or non-human materials do not have biological markers that cause human cells to recognize the synthetic material as human biological tissue. As a result, the human body can form scar tissue or a fibrous capsule around synthetic or non-human material as a defense mechanism, which can lead to complications. Such rejection may be less likely to occur when fibrous connective tissue such as the tibialis tendon is used in a sacrocolpopexy procedure, because the fibrous connective tissue does include biological markers that lead the patient’s native tissue to “recognize” the biologic implant. Furthermore, biologic tissues derived from human fibrous connective tissue can exhibit tissue–implant interaction characteristics that differ from those of permanent synthetic or non-human materials. When processed for implantation, such biologic tissues can retain structural and biochemical features that are compatible with host tissue environments.

Sixth, using human tissue may decrease a likelihood of infection as compared with solutions involving synthetic material. Bacteria can form biofilms on synthetic materials. Biofilms on implanted synthetic materials can be resistant or tolerant to antibiotics and host immune defenses, can be difficult to detect and eradicate, and in some cases may require removal of the implant. Furthermore, the body of the patient cannot typically vascularize implanted synthetic material, and thus the implanted synthetic material cannot fight infection. Using fibrous connective tissue such as tibialis tendon in a pelvic suspension procedure can provide material characteristics that differ from synthetic implants, including differences in surface properties and interaction with surrounding tissue, which may influence susceptibility to bacterial colonization. Additionally, biologic tissues derived from human fibrous connective tissue can differ from permanent synthetic materials with respect to surface characteristics and integration with surrounding tissue.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIGS. 1A-1D illustrate a system including one or more fibrous connective tissue samples that are attached to an upper portion of a vaginal canal and a sacrum of the patient.

FIGS. 2A-2B illustrate a system including two fibrous connective tissue samples.

FIG. 3 illustrates locations for incisions in a minimally invasive pelvic suspension procedure.

FIGS. 4-5 illustrate example configurations of a tibialis tendon sample that can be used as a fibrous connective tissue sample.

FIGS. 6-8 illustrate a tendon-based tissue sample that can be used to create a graft for use in a laparoscopic sacrocolpopexy procedure.

FIG. 9 is a flow diagram illustrating an example technique for performing a pelvic suspension procedure using one or more fibrous connective tissue samples.

### DETAILED DESCRIPTION

This disclosure describes systems, constructs, and methods for restoring pelvic support using one or more fibrous connective tissue samples configured for attachment to pelvic tissue at anterior and posterior pelvic support sites and secured to a fixation site at or adjacent to the sacral promontory, including fixation to the anterior longitudinal ligament. In some embodiments, the fibrous connective tissue samples comprise human allograft tissue that may be processed to reduce cellular content while preserving structural integrity. The disclosed constructs can be implanted using robotic, laparoscopic, or open surgical techniques and can be used in pelvic suspension procedures involving gynecologic, urologic, or colorectal anatomy. One example of a pelvic suspension procedure is a sacrocolpopexy.

In one embodiment, a biologic method for sacrocolpopexy involves using fibrous connective tissue—specifically, decellularized cadaveric tibialis tendons—to restore pelvic support. The tendon grafts can be affixed between the anterior and posterior vaginal walls and the sacral promontory, and can be implanted using robotic, laparoscopic, or open techniques. This approach can offer strong biocompatibility, durable support, and can serve as a mesh-free alternative for patients with prior complications or those seeking non-synthetic solutions.

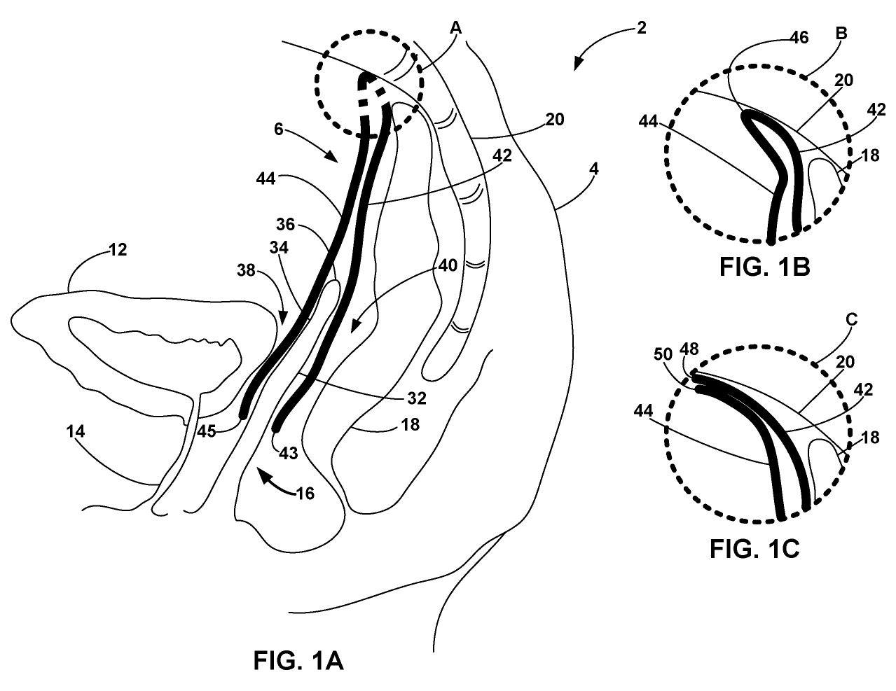

Referring now to FIG. 1A, some embodiments of a system 2 for treating a pelvic organ prolapse of a patient 4 involve one or more fibrous connective tissue samples 6 that form a graft to provide support to one or more pelvic organs. In some examples, each of the one or more fibrous connective tissue samples 6 can include a tibialis tendon sample. A clinician can use laparoscopic and/or robotic surgical procedures to implant the one or more fibrous connective tissue samples 6 into a body of the patient 4 to treat the pelvic organ prolapse, these laparoscopic and/or robotic surgical procedures being less invasive than open surgery methods where a large incision is made in the abdomen. When the one or more fibrous connective tissue samples 6 are implanted within the body of the patient 4, the one or more fibrous connective tissue samples 6 can maintain the pelvic organ in position. This maintained position sometimes approximates a position of the pelvic organ before a condition or event that led to the pelvic organ prolapse, such as a hysterectomy. As discussed in greater detail, during a sacrocolpopexy procedure, the pelvic organ is moved to a position (e.g., by a clinician or a robot), and the pelvic organ is maintained in the position using the one or more fibrous connective tissue samples 6.

Patient 4 has one or more pelvic organs which may include a bladder 12, urethra 14, vagina 16, and rectum 18. The patient 4 also has a sacrum 20, which forms a posterior bony portion of the pelvis and can serve as a stable anatomical landmark for fixation relative to the pelvic anatomy. In many patients, the vagina 16 can be located between the bladder 12 and the rectum 18 and at least part of the rectum 18 can be located between the vagina 16 and the sacrum 20. Patient 4 can have a pelvis that defines a pelvic space including the bladder 12, vagina 16, and rectum 18 such that the bladder 12, vagina 16, and rectum 18 occupy locations within the pelvic space relative to each other. The sacrum 20 can help to define at least part of the pelvic space. This means that the bladder 12, vagina 16, and rectum 18 can be positioned within a pelvic space defined in part by the sacrum 20 and surrounding pelvic structures.

In some cases, the vagina 16 includes a posterior vaginal wall 32, an anterior vaginal wall 34, and the vaginal apex or vaginal vault 36. In anatomy, the term “anterior” can refer to the front of the body (e.g., abdominal wall) and “posterior” can refer to the rear of the body such as the buttocks. This means that the anterior vaginal wall 34 includes a portion of the vaginal wall that is closest to the front of the body and the posterior vaginal wall 32 refers to a portion of the vaginal wall that is closest to the rear of the body. In some cases, the anterior vaginal wall 34 generally corresponds to a portion of the vaginal wall oriented toward the anterior pelvic compartment, while the posterior vaginal wall 32 corresponds to a portion oriented toward the posterior pelvic compartment. Vaginal vault 36 includes an upper portion of the vaginal canal, including the apex of the vaginal canal. In a vaginal vault prolapse, the vaginal vault 36 (or vaginal vault apex) can collapse or prolapse into the vaginal canal, leading to complications.

FIG. 1A and the preceding description illustrate vaginal anatomy and associated pelvic support sites as one example, but this disclosure is not limited to vaginal support procedures such as sacrocolpopexy. In some embodiments, pelvic support sites may include tissue associated with other pelvic organs, including colorectal anatomy such as the rectum. In such embodiments, one or more fibrous connective tissue samples may be attached to colorectal tissue or associated supporting structures and secured to a fixation site at or adjacent to the sacral promontory, including fixation to the anterior longitudinal ligament, to provide pelvic suspension. In such embodiments where the patient still has an intact uterus, one or more fibrous connective tissue samples may be attached to the anterior or posterior position of the uterus associated supporting structures and secured to a fixation site at or adjacent to the sacral promontory, including fixation to the anterior longitudinal ligament, to provide uterine/pelvic suspension.

The posterior vaginal wall 32 can be positioned anterior to the rectum 18. In some examples, the posterior vaginal wall 32 and the rectum 18 can be separated by a thin layer of connective tissue often referred to as the rectovaginal fascia. This connective tissue can provide support and can help to maintain a normal position of vagina 16 and the rectum 18. During childbirth, for example, the rectum and vaginal wall can move in coordination, with the rectovaginal fascia allowing flexibility while preventing the rectum from bulging into the vaginal canal. The anterior vaginal wall 34 can be positioned posterior to the bladder 12 and urethra 14, separated by a layer of connective tissue often referred to as the pubocervical fascia. Connective tissue layers such as the rectovaginal fascia and the pubocervical fascia can define anatomical planes between adjacent pelvic organs and may be encountered during pelvic suspension procedures such as sacrocolpopexy. The pubocervical fascia can support the bladder 12 and helps maintain a normal position of the bladder 12 relative to the vagina 16. The anterior vaginal wall 34 can provide structural support during activities such as urination and physical movement, while the pubocervical fascia ensures that the bladder 12 does not protrude into the vaginal canal.

In some cases, the vaginal vault 36 occupies a position that is superior to the bladder 12 within the pelvic cavity. The vaginal vault 36 refers to an upper, apical portion of the vagina 16, to which the uterus can be attached before removal. After a hysterectomy, the vaginal vault 36 can become the apex of the vaginal canal. The bladder 12 sits in front and below the level of the vaginal apex in most standing or upright positions. However, because the vaginal vault 36 can be suspended by ligaments that were once connected to the uterus (e.g., the uterosacral and cardinal ligaments), the vaginal vault 36 lies somewhat superior to the bladder 12, especially when the patient 4 is supine. Although the bladder 12 is anterior to the vagina 16, the vaginal vault 36 can extend higher in the pelvic cavity than the bladder 12 depending on posture and surgical anatomy.

Pelvic suspension procedures can be performed when one or more pelvic organs lack sufficient support relative to surrounding pelvic anatomy. In some clinical contexts, pelvic suspension can be performed following surgical alteration of pelvic structures or in association with changes to connective tissue or muscular support within the pelvis. In such cases, one or more fibrous connective tissue samples 6 can be attached to selected pelvic support sites and fixed to a stable anatomical landmark to provide pelvic suspension.

A pelvic organ prolapse can occur when a pelvic organ loses support and drops relative to its normal position. In some examples, pelvic organ prolapse occurs when an upper portion of the vagina 16 (e.g., the vaginal vault 36) loses support, causing the vaginal canal to collapse towards the vaginal opening. This can occur after a hysterectomy, where part or all of the uterus is removed, thus decreasing or reducing structural support of the vaginal vault 36. For example, when the uterus is removed without adequate support of the surrounding pelvic structures (e.g., the vagina 16), this can lead to pelvic organ prolapse because the uterus serves as a central support structure within the pelvis. When the uterus is removed, the ligaments and connective tissues that anchored the vagina 16 to the uterus (e.g., the uterosacral and cardinal ligaments) can become weakened or are not properly reattached, reducing support for the vaginal cuff. Over time, this loss of structural support can cause the pelvic organs to shift downward or bulge into the vaginal canal, resulting in prolapse. Risk of prolapse can increase when pelvic floor muscles are also weakened, such as from childbirth, aging, or chronic straining.

In some examples, a clinician can perform a sacrocolpopexy procedure to restore support to an upper end of the vagina 16 to treat prolapse by restoring support that was lost because of one or more patient conditions or procedures (e.g., hysterectomy). By restoring support, the clinician can return vagina 16 to a former position before support was lost because of the one or more patient conditions or procedures. Although vaginal prolapse is a common prolapse that can be treated using the methods described herein, this disclosure is not limited to procedures for treating a vaginal vault prolapse. In some cases, techniques described herein can be used to provide pelvic suspension involving tissue associated with other pelvic organs, including colorectal, bladder, or urethral anatomy, using attachment and fixation principles consistent with those described herein.

In some embodiments, the systems and methods described herein can be used in minimally invasive pelvic suspension procedures, including laparoscopic or robotic-assisted approaches. In such embodiments, access to the pelvic cavity can be obtained through one or more abdominal access sites sized to accommodate visualization and surgical instrumentation. Visualization of pelvic anatomy can be provided using an endoscopic imaging device, allowing placement and attachment of one or more fibrous connective tissue samples at selected pelvic support sites and fixation to a stable anatomical landmark as described herein. In other embodiments, the systems and methods described herein may be implemented using an open surgical approach.

In use, in some embodiments, a clinician can perform a method for performing a laparoscopic and/or robotic sacrocolpopexy, to treat pelvic organ prolapse in patient 4. In a laparoscopic sacrocolpopexy procedure to treat a vaginal vault prolapse, the clinician can make small incisions in the abdomen of patient 4 through which a camera (e.g., a laparoscope) can be inserted. The clinician can make these incisions manually in some embodiments, or in other embodiments, the clinician can direct a robot to make these incisions. This camera provides direct visualization for the clinician to observe interior pelvic anatomy of patient 4 on a screen, obviating a need for the clinician to make a larger incision in the abdomen to provide visualization. The incisions in the patient’s anatomy are sized to fit the diameter of the laparoscope and other narrow elongated tools and are much smaller than the larger incision made in an open surgery procedure. Therefore, the laparoscopic procedure can be less invasive than the open procedure.

Under direct visualization by a camera, the clinician can dissect tissue to expose the vagina 16 (including the vaginal vault) and the sacral promontory. Dissection can involve, in some examples, dissecting the anterior vaginal wall 34 from the bladder 12 and dissecting the posterior vaginal wall 32 from the rectum 18. In some embodiments, dissecting the anterior vaginal wall 34 from the bladder 12 involves cutting the pubocervical fascia that connects the anterior vaginal wall 34 and the bladder 12 to create a space 38 between the anterior vaginal wall 34 and the bladder 12. In some embodiments, dissecting the posterior vaginal wall 32 from the rectum 18 involves cutting the rectovaginal fascia to create a space 40 between the posterior vaginal wall 32 and the rectum 18. Dissection can be performed, in some cases, using Metzenbaum scissors, a scalpel, blunt dissection tools, or energy devices delivered to the dissection site via one or more small incisions in the abdomen of patient 4.

In some embodiments, a fixation site at or adjacent to the sacral promontory can be prepared for attachment of one or more fibrous connective tissue samples 6. Preparation of the fixation site can include exposure of the anterior longitudinal ligament of the sacrum 20 using one or more tissue separation techniques while preserving surrounding anatomical structures. Fibrous connective tissue samples 6 can be secured to the fixation site using sutures, anchors, or other fixation mechanisms. The samples may extend from pelvic tissue to the fixation site along a path that is anterior to, adjacent to, or lateral to the sacrum 20 in some examples.

The clinician can skeletonize the sacral promontory to create an attachment location for the one or more fibrous connective tissue samples 6 in some cases. For example, in a laparoscopic sacrocolpopexy procedure, skeletonization of the sacral promontory involves carefully exposing the anterior longitudinal ligament over the sacrum 20 to allow secure attachment while avoiding injury to nearby vital structures. In some cases, the clinician can identify anatomical landmarks such as the aortic bifurcation, right common iliac artery, and left common iliac vein. Dissection can be performed to the right of the midline, medial to the right common iliac artery, where the promontory lies most accessible. Using a combination of blunt and sharp dissection, the clinician can incise and mobilize the peritoneum overlying the sacral promontory. When the anterior longitudinal ligament is cleanly exposed, ensuring a bloodless field, the ligament is ready for the attachment of the one or more fibrous connective tissue samples 6, completing skeletonization.

Following preparation of the pelvic tissue and fixation site (e.g., including dissection and skeletonization), one or more fibrous connective tissue samples 6 can be introduced into patient 4 through one or more access openings in the abdominal wall. In some embodiments, the fibrous connective tissue samples 6 comprise human connective tissue prepared for implantation and configured for use in a minimally invasive or open pelvic suspension procedure. In some examples, the one or more fibrous connective tissue samples 6 can represent tibialis tendon samples (e.g., the anterior tibialis tendon and/or or the posterior tibialis tendon) harvested from cadaveric donors and prepared for use in the laparoscopic and/or robotic sacrocolpopexy procedure. The one or more fibrous connective tissue samples 6 may, in some examples, be sized as elongate, thin strips having a width less than a length and a depth less than the width. In some examples, each of the one or more fibrous connective tissue samples 6 can include a first end and a second end, the first end being wider than the second end, but this is not required. In some examples, each of the one or more fibrous connective tissue samples 6 can have substantially uniform width along a length of the sample.

In some embodiments, the fibrous connective tissue samples 6 comprise tendon tissue selected for its mechanical strength, collagen organization, and suitability for load-bearing suspension within pelvic anatomy. Tendon tissue can be composed primarily of dense, regularly aligned Type I collagen fibers arranged in parallel bundles, providing resistance to elongation under sustained tensile load. In some embodiments, fibrous connective tissue samples 6 can comprise tibialis tendon tissue, which exhibits a highly organized collagen architecture adapted for force transmission and is suitable for load-bearing suspension within pelvic anatomy when secured between pelvic tissue attachment sites and a fixation site at or adjacent to the sacrum. The tibialis tendon is a strong, fibrous connective tissue that attaches a tibialis muscle to the medial cuneiform and first metatarsal bones of the foot. The tibialis tendon plays a role in dorsiflexion (e.g., lifting the foot upward) and inversion (e.g., turning the sole of the foot inward). The tibialis tendon can be composed primarily of dense regular connective tissue, rich in type I collagen fibers. These collagen fibers can be highly organized in parallel bundles, which provides strength and durability. Collagen alignment of the tibialis tendon can provide exceptional tensile strength, allowing the tibialis tendon to resist stretching while transmitting force efficiently. Both of the anterior tibialis tendon and the posterior tibialis tendon are suitable for use in the one or more fibrous connective tissue samples 6.

Much of the strength and durability of the tibialis tendon can be attributed to the extracellular matrix (ECM). The ECM of the tibialis tendon is a network of proteins and carbohydrates that surrounds the cells of the tibialis tendon. In some cases, the ECM of the tibialis tendon is a dense, fibrous network primarily including type I collagen, which provides high tensile strength and resistance to stretching. The collagen fibers can be aligned in parallel bundles to support unidirectional force transmission. The organized collagen architecture facilitates directional force transmission and dimensional stability when the tissue is placed under sustained tension. Interspersed within the ECM are proteoglycans and glycosaminoglycans (GAGs), such as decorin and biglycan, which help to maintain hydration and contribute to viscoelastic properties, allowing the tendon to absorb and release mechanical energy. Elastin fibers are also present in some cases, providing limited elasticity. Structural components of the ECM can contribute to hydration and limited elasticity, allowing the tissue to accommodate physiological movement while maintaining structural support. In embodiments in which the tendon tissue is decellularized, cellular components may be substantially removed while the ECM structure is preserved, thereby maintaining the mechanical characteristics of the tissue while reducing cellular content.

Because of the strength and durability of the tibialis tendon, it can be beneficial for the clinician to use tibialis tendon samples as the one or more fibrous connective tissue samples 6. The tibialis tendon has structural characteristics that are suitable for load-bearing suspension within pelvic anatomy. During a pelvic suspension procedure, a clinician may secure portions of the fibrous connective tissue samples 6 to pelvic tissue at one or more anterior and posterior support sites along the vagina 16, including at or near the vaginal vault 36.

As described above, preparation of the anterior vaginal wall 34 and posterior vaginal wall 32 can create tissue planes that permit placement of the fibrous connective tissue samples 6 between the vagina 16 and adjacent pelvic organs. In some embodiments, a portion of the fibrous connective tissue samples 6 may be positioned between the posterior vaginal wall 32 and the rectum 18, and another portion may be positioned between the anterior vaginal wall 34 and the bladder 12. One or more portions of the fibrous connective tissue samples 6 may additionally be secured to a fixation site at or adjacent to the sacrum 20, including fixation to the anterior longitudinal ligament. In this manner, the fibrous connective tissue samples 6 can function as a suspension construct configured to support pelvic anatomy following loss of native support.

In some examples, the clinician can suture the one or more fibrous connective tissue samples 6 including tibialis tendon samples to the upper portion of the vagina 16 including the vaginal vault 36. As described above, the anterior vaginal wall 34 can be dissected from the bladder 12, and the posterior vaginal wall 32 can be dissected from the rectum 18, thus enabling the clinician to suture the one or more fibrous connective tissue samples 6 directly to the posterior vaginal wall 32 between the posterior vaginal wall 32 and the rectum 18 and suture the one or more fibrous connective tissue samples 6 directly to the anterior vaginal wall 34 between the anterior vaginal wall 34 and the bladder 12. In some examples, the clinician can suture the one or more fibrous connective tissue samples 6 to the sacral promontory proximate the sacrum 20. For example, the clinician can suture the one or more fibrous connective tissue samples 6 directly to the anterior longitudinal ligament over the sacrum 20, effectively suspending the vagina 16 in its normal anatomical position. This minimally invasive approach can restore pelvic support while minimizing recovery time, postoperative pain, and risk of complications as compared with open surgery.

In some embodiments, tibialis tendon tissue may be used as the one or more fibrous connective tissue samples 6 due to its structural characteristics and suitability for load-bearing suspension within pelvic anatomy. During a pelvic suspension procedure, a clinician can secure portions of the fibrous connective tissue samples 6 to pelvic tissue at one or more anterior and posterior support sites along vagina 16, including at or near the vaginal vault 36. As described above, preparation of the anterior vaginal wall 34 and posterior vaginal wall 32 can create tissue planes that permit placement of the fibrous connective tissue samples 6 between the vagina 16 and adjacent pelvic organs. For example, a portion of the fibrous connective tissue samples 6 may be positioned between the posterior vaginal wall 32 and the rectum 18, and another portion may be positioned between the anterior vaginal wall 34 and the bladder 12. One or more portions of the fibrous connective tissue samples 6 may additionally be secured to a fixation site at or adjacent to the sacrum 20, including fixation to the anterior longitudinal ligament. In this manner, the fibrous connective tissue samples 6 can function as a suspension construct configured to support pelvic anatomy following loss of native support.

In a laparoscopic sacrocolpopexy procedure, suturing the one or more fibrous connective tissue samples 6 to the posterior vaginal wall 32, the anterior vaginal wall 34, and the anterior longitudinal ligament over the sacrum 20 can involve a precise laparoscopic technique to ensure durable support while avoiding injury to surrounding structures. After mobilizing the bladder 12 and rectum 18 to expose the posterior and anterior vaginal walls 32, 34, the one or more fibrous connective tissue samples 6 can be introduced into the pelvic area of patient 4 through a trocar. In some embodiments, the one or more fibrous connective tissue samples 6 can be sutured to the posterior vaginal wall 32 and the anterior vaginal wall 34 using delayed absorbable sutures. In some embodiments, 2/0 or 3/0 sutures can be placed in a continuous or interrupted fashion to secure the anterior fibrous connective tissue portion 44 to the anterior vaginal wall 34 and to secure the posterior fibrous connective tissue portion 42 to the posterior vaginal wall 32.

In some embodiments, fibrous connective tissue samples 6 can be configured to include distinct portions oriented for attachment to anterior and posterior pelvic support sites. During a pelvic suspension procedure, posterior fibrous connective tissue portion 42 of the fibrous connective tissue samples 6 may be secured to posterior pelvic tissue, including the posterior vaginal wall 32, and an anterior fibrous connective tissue portion 44 may be secured to anterior pelvic tissue, including the anterior vaginal wall 34. Fibrous connective tissue samples 6 may be introduced into the pelvic cavity through an access port and positioned within prepared tissue planes adjacent to vagina 16. Attachment of the anterior and posterior portions can be performed using one or more fixation techniques selected by the clinician, including suturing or other tissue fixation methods suitable for minimally invasive or open procedures. A separate portion of the fibrous connective tissue samples 6 may be secured to a fixation site at or adjacent to the sacrum 20, including fixation to the anterior longitudinal ligament, such that the anterior and posterior portions extend from the fixation site toward their respective pelvic tissue attachment locations. In this configuration, the fibrous connective tissue samples 6 function as a suspension construct configured to support pelvic anatomy.

In performing the suturing, the clinician can make full-thickness bites while avoiding penetration into the vaginal epithelium. Using laparoscopic and/or robotic instruments with fine control, the clinician can suture under direct visualization, angling a needle to pass deeply enough for a secure hold but shallow enough to stay just beneath the epithelium. Tactile feedback can be limited during a laparoscopic procedure, so visual cues and experience are critical in maintaining the correct depth. Suturing in this way can ensure strong fixation that avoids epithelial violation, which could otherwise increase the risk of mesh exposure, erosion, or postoperative discomfort. In some embodiments, fixation may include suturing techniques that engage sufficient tissue depth to provide secure attachment while minimizing penetration of adjacent epithelial surfaces. Attachment of the fibrous connective tissue samples 6 is not limited to suturing. In performing attachment of the one or more fibrous connective tissue samples 6 to pelvic tissue and to the fixation site, the clinician may employ any suitable fixation method. The fixation approach can provide durable suspension of the pelvic tissue while reducing the risk of unintended tissue injury, exposure, or postoperative complications.

As described above, delayed absorbable sutures can be used to secure the anterior fibrous connective tissue portion 44 to the anterior vaginal wall 34 and to secure the posterior fibrous connective tissue portion 42 to the posterior vaginal wall 32. Absorbable sutures are surgical threads designed to break down and be absorbed by the body over time, obviating a need for removal. Absorbable sutures can be made from synthetic polymers such as polyglycolic acid (PGA), polylactic acid (PLA), or polydioxanone (PDS), or from processed natural materials such as catgut. These sutures can maintain tensile strength long enough to support tissue during the critical healing period, after which absorbable sutures are gradually hydrolyzed or enzymatically digested and absorbed by surrounding tissues. Using absorbable sutures in grafts of human tissue can be beneficial because absorbable sutures provide temporary mechanical support while minimizing long-term foreign body presence, which reduces a risk of chronic inflammation, infection, and erosion. In grafting procedures, such as a laparoscopic sacrocolpopexy procedure to attach one or more fibrous connective tissue samples 6 to the posterior vaginal wall 32 and the anterior vaginal wall 34, absorbable sutures can allow the host tissue to integrate and heal without interference from permanent materials that could provoke immune responses or impede remodeling. In some embodiments, anterior and posterior portions of the fibrous connective tissue samples 6 may be secured to corresponding pelvic tissue attachment sites using any suitable fixation materials and techniques selected by the clinician. Fixation materials can provide temporary or longer-term mechanical support based on anatomical considerations, surgical approach, and desired support characteristics. Selection of fixation materials and techniques does not limit the configuration or function of the fibrous connective tissue samples 6, and the fibrous connective tissue samples 6 are not dependent on a particular fixation strategy to provide pelvic suspension.

Another benefit of using tibialis tendon samples as the one or more fibrous connective tissue samples 6 is that the tibialis tendon includes an end that can fan out to a greater width as compared with a width of the rest of the tendon sample. That is, the tibialis tendon sample can include a portion having an increased width relative to other portions of the tendon. This widened portion can provide an increased surface area for attachment to pelvic soft tissue, such as tissue at or adjacent to the vaginal vault 36. This allows tension from the one or more fibrous connective tissue samples 6 to be distributed to a greater surface area of the vaginal vault 36, avoiding a situation where a small surface area bears a large portion of the strain. Increasing the attachment surface area can facilitate suturing to compliant tissue and distribute tensile loads across a broader region of tissue. In some embodiments, the widened portion corresponds to a naturally flared region of the tendon, including a region proximate a musculotendinous junction, although other configurations are contemplated.

In some cases, the end of the tibialis tendon that is connected to muscle in the cadaveric donor is the end of the tibialis tendon that can fan out to a larger surface area.

In some embodiments, the fibrous connective tissue sample includes regions of differing geometry selected to correspond to distinct anatomical attachment sites having different mechanical and structural characteristics. A first region of the tissue sample may include an expanded or fanned configuration that increases surface area for attachment to compliant pelvic soft tissue, such as the vaginal vault or associated fascial structures. A second region of the tissue sample may include a reduced, elongated, or substantially tubular configuration adapted for attachment to a ligamentous fixation site, such as the anterior longitudinal ligament of the sacrum, which is dense and linear in nature. The differing geometries are intentionally selected to accommodate the anatomy and mechanical behavior of the respective attachment sites rather than representing arbitrary variations in graft shape.

The expanded and reduced regions of the fibrous connective tissue sample are not interchangeable. Orienting the expanded region toward a ligamentous fixation site or orienting the reduced region toward compliant pelvic soft tissue would result in suboptimal fixation due to incompatibility between graft geometry and tissue structure. Accordingly, the tissue sample is configured such that each region cooperates with its corresponding anatomical attachment site to distribute load in a manner consistent with native tissue mechanics. This coordinated relationship between graft geometry and anatomical fixation sites is not taught or suggested by prior synthetic mesh constructs or uniform graft materials and provides a non-obvious structural configuration.

After securing the one or more fibrous connective tissue samples 6 to the posterior vaginal wall 32 and the anterior vaginal wall 34, the one or more fibrous connective tissue samples 6 can be tension-free aligned toward the sacral promontory and sutured (or otherwise attached) to the exposed anterior longitudinal ligament over the sacrum 20 using permanent nonabsorbable sutures. In some embodiments, nonabsorbable braided or monofilament material can be used, but other fixation modalities may be used in some embodiments. This fixation can provide strong apical support by anchoring vagina 16 to a stable point on the spine. In some cases, peritoneum can then be closed over the one or more fibrous connective tissue samples 6 to reduce a likelihood of bowel adhesions and internal herniation, completing the placement and reinforcement of the one or more fibrous connective tissue samples 6. Following fixation, peritoneal tissue may be repositioned or closed over at least a portion of the fibrous connective tissue samples 6 to restore peritoneal continuity and complete placement of the support construct.

A peritoneum is a thin, transparent serous membrane that lines the abdominal cavity and covers many of the abdominal organs, including a parietal layer lining the abdominal wall and a visceral layer covering the organs. The peritoneum can play a critical role in protecting internal structures, reducing friction between organs with lubricating serous fluid, and serving as a barrier against infection. In embodiments where one or more fibrous connective tissue samples 6 are placed inside the body of patient 4, covering the one or more fibrous connective tissue samples 6 with peritoneum offers several benefits by physically separating the grafts from mobile abdominal organs and reducing a risk of adhesions, erosion, and fistula formation. Additionally, the peritoneal covering can help anchor and protect the graft during early healing, support neovascularization, and minimize immune reaction by isolating the foreign material from direct exposure to the peritoneal cavity, thereby enhancing biocompatibility and long-term integration. For example, following fixation of the one or more fibrous connective tissue samples 6, peritoneal tissue may be repositioned or closed over at least a portion of the fibrous connective tissue samples 6. Repositioning or closing the peritoneum over the fibrous connective tissue samples 6 can separate the samples from the peritoneal cavity and mobile abdominal structures. Such peritoneal management can be performed at the discretion of the clinician and may vary depending on surgical approach, anatomy, or clinical preference.

The one or more fibrous connective tissue samples 6 can be positioned to provide adequate tension and support for the vaginal vault 36 without causing overcorrection or strain. For example, one or more fibrous connective tissue samples 6 can be configured and positioned to extend between pelvic tissue of the patient 4 and a fixation site at or adjacent to the sacral promontory. This can be achieved through placement of sutures and sizing of the one or more fibrous connective tissue samples 6. In some embodiments, a first portion of the one or more fibrous connective tissue samples 6 extends from the posterior vaginal wall 32 toward the sacrum 20, and a second portion of the one or more fibrous connective tissue samples 6 extends from the anterior vaginal wall 34 toward the sacrum 20. For example, the one or more fibrous connective tissue samples 6 can be sized to extend from the posterior vaginal wall 32 to the anterior longitudinal ligament over the sacrum 20 and extend from the anterior vaginal wall 34 to the anterior longitudinal ligament over the sacrum 20 such that the one or more fibrous connective tissue samples 6 provide enough tension for maintaining the vaginal vault 36 in place without applying too much tension to cause strain or overcorrection of the vaginal vault 36.

In some embodiments, robots can aid in performing a laparoscopic sacrocolpopexy to correct a vaginal vault prolapse. Robots can provide enhanced visualization, precision, and dexterity. For example, a clinician can use a robotic surgical system to control instruments inserted through small abdominal incisions. The robotic system can provide three-dimensional, high-definition magnified visualization, allowing the clinician to clearly see pelvic structures including the sacral promontory, vaginal vault, and surrounding organs. The robotic arms can mimic the surgeon’s hand movements but with greater range of motion and tremor filtration, which is particularly helpful when dissecting delicate tissue planes like the rectovaginal, vesicovaginal, and pubocervical spaces, and suturing the one or more fibrous connective tissue samples 6 to the posterior vaginal wall 32, the anterior vaginal wall 34, and the sacral promontory. This disclosure is not limited to using robots to aid in performing a procedure. The procedures described herein may be performed using open, laparoscopic, or robot-assisted surgical approaches.

Some sacrocolpopexy procedures use synthetic polypropylene mesh to provide support. Complications such as erosion, migration, infection, and chronic inflammation can occur when using a synthetic polypropylene mesh in a sacrocolpopexy procedure. Because synthetic polypropylene mesh is not made of human tissue, this can result in the synthetic polypropylene mesh migrating or eroding human tissue over time. For example, scar tissue can form around a synthetic polypropylene mesh. Additionally, or alternatively, the synthetic polypropylene mesh can encroach into unwanted areas, causing pain or discomfort. Infections can occur more frequently with synthetic polypropylene mesh, because this material is prone to gathering bacteria due to the lack of biologic response from the material. In any case, when a sacrocolpopexy procedure involves using cadaveric tissue such as cadaveric anterior or posterior tibialis tendons as the one or more fibrous connective tissue samples 6, this can offer a biocompatible alternative that reduces risks while maintaining efficacy and structural integrity.

In some embodiments, the one or more fibrous connective tissue samples 6 can include a posterior fibrous connective tissue portion 42 that terminates at a posterior lower end 43 and an anterior fibrous connective tissue portion 44 that terminates at an anterior lower end 45. A lower part of the posterior fibrous connective tissue portion 42 can be sutured (or otherwise attached) to an upper portion of the posterior vaginal wall 32, and an upper part of the anterior fibrous connective tissue portion 44 can be sutured (or otherwise attached) to an upper part of the anterior vaginal wall 34. Because the anterior vaginal wall 34 can be dissected from bladder 12 and the posterior vaginal wall 32 can be dissected from the rectum 18, this means that a space 38 exists between the bladder 12 and the anterior vaginal wall 34 and a space 40 exists between the rectum 18 and the posterior vaginal wall 32. The posterior and anterior fibrous connective tissue portions 42, 44 may be positioned within the spaces for attachment to pelvic tissue.

In some embodiments, the posterior fibrous connective tissue portion 42 is sutured or otherwise attached to the posterior vaginal wall 32 such that the posterior fibrous connective tissue portion 42 is sized to fit within the space 40 between the rectum 18 and the posterior vaginal wall 32. In some embodiments, the anterior fibrous connective tissue portion 44 is sutured or otherwise attached to the anterior vaginal wall 34 such that the anterior fibrous connective tissue portion 44 is sized to fit within the space 38 between the anterior vaginal wall 34 and the bladder 12. As depicted in FIG. 1A, the posterior fibrous connective tissue portion 42 can extend along an upper portion of the posterior vaginal wall 32, terminating at posterior lower end 43. Similarly, the anterior fibrous connective tissue portion 44 can extend along an upper portion of the anterior vaginal wall 34 terminating at anterior lower end 45.

The posterior fibrous connective tissue portion 42 and/or the anterior fibrous connective tissue portion 44 can be secured to a ligamentous structure associated with the sacrum 20, such as the anterior longitudinal ligament, at an attachment region A. The anterior longitudinal ligament extends along the anterior aspect of the sacrum and provides a relatively fixed anatomical structure suitable for anchoring supportive constructs within the pelvis. In some embodiments, attachment of the posterior fibrous connective tissue portion 42 and/or the anterior fibrous connective tissue portion 44 at the attachment region provides suspension support to the vaginal vault 36 relative to the sacrum 20. This configuration can reposition pelvic tissue toward an anatomically appropriate orientation and can supplement or replace native support structures that may be absent or compromised.

In a sacrocolpopexy procedure, the posterior fibrous connective tissue portion 42 and/or the anterior fibrous connective tissue portion 44 can be sutured to an anterior longitudinal ligament over the sacrum 20 because this ligament provides a strong, stable, and immobile anchoring point deep in the pelvis. Located along the front of the vertebral column, the anterior longitudinal ligament is tightly bound to the sacral promontory and is well-suited to support the weight and movement of the pelvic organs. By attaching the posterior fibrous connective tissue portion 42 and/or the anterior fibrous connective tissue portion 44 to the anterior longitudinal ligament, the clinician can elevate and suspend the vaginal vault 36 in a natural anatomical position. This anchoring method can correct a pelvic organ prolapse that occurs when the vaginal vault 36 loses support (e.g., because of a hysterectomy). This anchoring method can further offer long-term durability and mimics the original support once provided by uterosacral and cardinal ligaments (e.g., before the hysterectomy), helping to restore normal vaginal orientation and prevent recurrence.

Referring now to FIGS. 1A-1C, the one or more fibrous connective tissue samples 6 can include the posterior fibrous connective tissue portion 42 and the anterior fibrous connective tissue portion 44. The posterior fibrous connective tissue portion 42 and the anterior fibrous connective tissue portion 44 can be attached to a ligamentous structure of the sacrum 20, such as the anterior longitudinal ligament, at an attachment region A so that the posterior fibrous connective tissue portion 42 and the anterior fibrous connective tissue portion 44 can cooperate to suspend the vaginal vault 36 in a normal anatomical position to correct a vaginal vault prolapse. For example, because the posterior fibrous connective tissue portion 42 is attached to the posterior vaginal wall 32 and the anterior fibrous connective tissue portion 44 is attached to the anterior vaginal wall 34, the posterior fibrous connective tissue portion 42 and the anterior fibrous connective tissue portion 44 apply approximately the same amount of tensile force to the vaginal canal. This means that supportive forces applied by the fibrous connective tissue samples 6 can be distributed across both anterior and posterior aspects of the vaginal vault 36. This cooperative configuration can reduce localized stress concentrations and promote stable suspension of the vaginal vault 36.

Referring now to FIG. 1B, in some embodiments the posterior fibrous connective tissue portion 42 and the anterior fibrous connective tissue portion 44 are formed from a single fibrous connective tissue sample 6 that is folded over itself at a folded portion 46. In this embodiment, the posterior fibrous connective tissue portion 42 and the anterior fibrous connective tissue portion 44 represent continuous portions of the same fibrous connective tissue sample 6. The folded portion 46 can be positioned at or adjacent to a sacral fixation site such that the single fibrous connective tissue sample 6 extends along the posterior vaginal wall 32, traverses toward the sacrum 20, and extends along the anterior vaginal wall 34. In some embodiments, the folded configuration allows the posterior fibrous connective tissue portion 42 and the anterior fibrous connective tissue portion 44 to cooperate as a continuous support construct when secured at the sacral fixation site, while maintaining continuity of the fibrous connective tissue sample 6. This arrangement can facilitate suspension of pelvic tissue using a single elongated tissue sample and may reduce the need for separate anterior and posterior graft components. This single fibrous connective tissue sample 6 depicted in the embodiment of FIG. 1B can represent a convenient sample to use in a sacrocolpopexy procedure, especially when the single fibrous connective tissue sample 6 includes a tibialis tendon sample. The tibialis tendon is sufficiently long to yield a sample that extends along the posterior vaginal wall 32, reaching the anterior longitudinal ligament over the sacrum 20, and extends back along the anterior vaginal wall 34. Using a single fibrous connective tissue sample 6 can provide extra strength and rigidity as the posterior fibrous connective tissue portion 42 and the anterior fibrous connective tissue portion 44 are connected together at the folded portion 46.

Referring now to FIG. 1C, in some embodiments the posterior fibrous connective tissue portion 42 and the anterior fibrous connective tissue portion 44 are each directly secured to a fixation site. For example, the posterior fibrous connective tissue portion 42 and the anterior fibrous connective tissue portion 44 can be two separate fibrous connective tissue samples 6 that are each secured to a fixation site such as a ligamentous structure of the sacrum 20 including the anterior longitudinal ligament. In some embodiments, the posterior fibrous connective tissue portion 42 and the anterior fibrous connective tissue portion 44 are each directly secured to the fixation site. In some examples, the posterior fibrous connective tissue portion 42 is sutured directly to the anterior longitudinal ligament over the sacrum 20 and the anterior fibrous connective tissue portion 44 is sutured to the anterior longitudinal ligament over the sacrum 20 through the posterior fibrous connective tissue portion 42.

In some embodiments, both of the posterior fibrous connective tissue portion 42 and the anterior fibrous connective tissue portion 44 are sutured directly to the fixation site such as a ligamentous structure of the sacrum 20 including the anterior longitudinal ligament. In other embodiments, one fibrous connective tissue sample may be secured in proximity to the other without requiring physical coupling between the samples. The posterior fibrous connective tissue portion 42 can terminate at a posterior upper end 48 and the anterior fibrous connective tissue portion 44 can terminate at an anterior upper end 50. It can be convenient in some cases to use two separate fibrous connective tissue samples 6 because this shortens a length of the two separate fibrous connective tissue samples 6 for easier maneuverability within the body of patient 4.

One part of a method for performing a sacrocolpopexy involves tendon preparation. For example, the one or more fibrous connective tissue samples 6 can include tibialis tendon samples that are harvested from cadaveric donors. Creating the one or more fibrous connective tissue samples 6, involves, in some embodiments, decellularization to remove cellular components, minimize immunogenicity, and preserve the ECM. In some examples, decellularizing a tendon tissue sample involves removing all cellular components including tenocytes, DNA, and cellular debris—while preserving the ECM, which provides the structural and mechanical integrity of the tendon. This process can include chemical, enzymatic, and mechanical methods. Detergents (e.g., Triton X-100, sodium dodecyl sulfate (SDS)) can be used to lyse and remove cells, and enzymes (e.g., deoxyribonuclease (DNase) and ribonuclease (RNase) can be used to degrade nucleic acids. Mechanical agitation or perfusion can be used to distribute agents throughout the tissue and wash away cellular remnants. To preserve collagen fibers, proteoglycans, and other ECM components, care can be taken to avoid overexposure during decellularization. This process can yield a cell-free scaffold that retains the native structure and mechanical properties of the tendon, suitable use as the one or more fibrous connective tissue samples 6.

When the one or more fibrous connective tissue samples 6 have been decellularized, these one or more samples can be sterilized and trimmed. The one or more connective tissue samples 6 can be trimmed to a length within a range from 30 centimeters (cm) to 50 cm (e.g., between 32 and 48 centimeters, 34 and 46 centimeters, 36 and 44 centimeters, between 38 and 42 centimeters, etc.) in the embodiment of FIG. 1B where there is a single connective tissue sample 6. The one or more samples 6 can each be trimmed to a length within a range from 15 cm to 25 cm (e.g., between 16 and 24 centimeters, 17 and 23 centimeters, 18 and 22 centimeters, between 19 and 21 centimeters, etc.) in the embodiment of FIG. 1C where there are two separate fibrous connective tissue samples 6. In some cases, a single tibialis tendon can yield a single connective tissue sample 6. In some cases, a single tibialis tendon can yield two or more connective tissue samples 6.

In some embodiments, the one or more fibrous connective tissue samples 6 are provided with native anatomical features substantially preserved, including one or more naturally flared end regions of a tibialis tendon. These flared end regions can provide increased surface area for attachment to soft tissue and can reduce localized stress concentrations when secured to the vaginal wall. In embodiments where a continuous fibrous connective tissue sample 6 is used, the flared end regions may be preserved through delivery to the operating room and selectively shaped, trimmed, or oriented by a clinician during surgery. In other embodiments, the fibrous connective tissue samples 6 may be pre-configured into a predetermined geometry, including a bifurcated configuration, prior to implantation. The selection and final configuration of the fibrous connective tissue samples 6 may be based on patient anatomy, fixation strategy, and desired load distribution, without departing from the scope of this disclosure.

In some embodiments, a sacrocolpopexy procedure involving one or more fibrous connective tissue samples 6 can be suitable for patients with pelvic organ prolapse, especially patients who have experienced prior complications with synthetic mesh, have experienced vaginal atrophy, have a thin vaginal epithelium, or have autoimmune conditions or sensitivities to synthetic materials. Because the one or more fibrous connective tissue samples 6 can provide the strength and durability of a synthetic mesh without causing one or more side effects associated with a synthetic mesh, it can be beneficial to use the one or more fibrous connective tissue samples 6 in a sacrocolpopexy procedure for at least some patients.

In an embodiment depicted in FIG. 1D, the posterior fibrous connective tissue portion 42 and the anterior fibrous connective tissue portion 44 are two separate fibrous connective tissue samples 6 that are connected at junction 51. In some cases, one of the posterior fibrous connective tissue portion 42 and the anterior fibrous connective tissue portion 44 has a longer length and the other one of the posterior fibrous connective tissue portion 42 and the anterior fibrous connective tissue portion 44 has a shorter length. In some embodiments, the posterior fibrous connective tissue portion 42 has the longer length, extending from posterior lower end 43 to upper end 52 and the anterior fibrous connective tissue portion 44 has the shorter length, extending from anterior lower end 45 to connect to posterior fibrous connective tissue portion 42 at junction 51. In some embodiments, the anterior fibrous connective tissue portion 44 has the longer length, extending from anterior lower end 45 to upper end 52 and the posterior fibrous connective tissue portion 42 has the shorter length, extending from posterior lower end 43 to connect to anterior fibrous connective tissue portion 44 at junction 51. In some cases, junction 51 can be formed prior to implantation. In some cases, junction 51 can be formed intraoperatively. Junction 51 can be fixed or adjustable.

In the configuration depicted in FIG. 1D, the one or more fibrous connective tissue samples 6 can include a Y-shaped graft including two branch portions and a trunk portion. In some cases, the two branch portions can attach to the posterior vaginal wall 32 and the anterior vaginal wall 34 and one trunk portion can attach to a fixation site at or adjacent to the sacral promontory, such as the anterior longitudinal ligament 22 over the sacrum 20. In some embodiments, the Y-shaped graft is formed from a single fibrous connective tissue sample 6, such as a tibialis tendon, wherein one branch portion extends continuously from one of the posterior vaginal wall 32 or the anterior vaginal wall 34 to the sacral fixation site, and the other branch portion joins the continuous portion at junction 51. In some embodiments, either branch portion can serve as the continuous portion, and the relative lengths of the branch portions can depend on patient anatomy or surgical approach. The Y-shaped configuration can be beneficial for several reasons, including that the Y-shaped configuration results in a strong graft from a single tendon sample from a single cadaveric donor. One reason that the Y-shaped configuration of FIG. 1D is beneficial is because the posterior fibrous connective tissue portion 42 or the anterior fibrous connective tissue portion 44 extends the entire length from the posterior vaginal wall 32 and the anterior vaginal wall 34 to the sacrum 20, and the other one of the posterior fibrous connective tissue portion 42 or the anterior fibrous connective tissue portion 44 attaches to the junction 51 to distribute force. This allows clinicians to create a graft from a single tibialis tendon sample, as a shorter length can be used to attach to one of the posterior vaginal wall 32 and the anterior vaginal wall 34. This configuration allows a single fibrous connective tissue sample 6 to be arranged to provide attachment at both anterior and posterior pelvic support sites while maintaining a single fixation region at the sacrum, without requiring the branch portions to be identical in length or orientation.

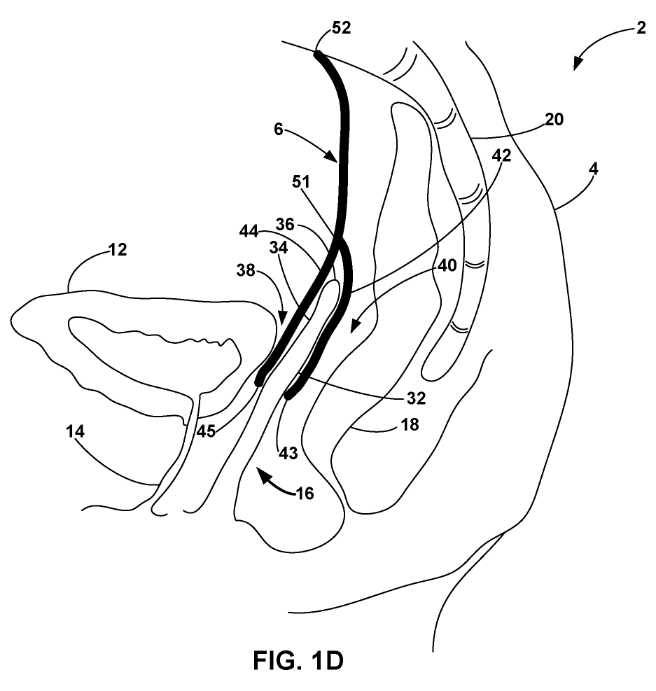

Referring now to FIGS. 2A-2B, an embodiment where two separate fibrous connective tissue samples 6 are used to suspend the vaginal vault 36 to the ligamentous structure of the sacrum 20, such as the anterior longitudinal ligament 22 is depicted. For example, as depicted in FIG. 2A, the two separate fibrous connective tissue samples 6 include a posterior fibrous connective tissue portion 42 and an anterior fibrous connective tissue portion 44. The posterior fibrous connective tissue portion 42 can be sutured or otherwise attached to the posterior vaginal wall 32 and the anterior fibrous connective tissue portion 44 can be sutured or otherwise attached to the anterior vaginal wall 34. The posterior fibrous connective tissue portion 42 can extend from a posterior lower end 43 to a posterior upper end 48 and the anterior fibrous connective tissue portion 44 can extend from an anterior lower end 45 to an anterior upper end 50.

As depicted in FIGS. 2A-2B, a lower part of the posterior fibrous connective tissue portion 42 can be sutured to the posterior vaginal wall 32 and a lower part of the anterior fibrous connective tissue portion 44 can be sutured to the anterior vaginal wall 34. An upper part of the posterior fibrous connective tissue portion 42 and an upper part of the anterior fibrous connective tissue portion 44 can be sutured to the anterior longitudinal ligament 22 in some embodiments. For example, the posterior fibrous connective tissue portion 42 can be sutured directly to the anterior longitudinal ligament 22 and the anterior fibrous connective tissue portion 44 can be sutured to the anterior longitudinal ligament 22 through the posterior fibrous connective tissue portion 42. That is, the posterior fibrous connective tissue portion 42 and/or the anterior fibrous connective tissue portion 44 can be placed on the anterior longitudinal ligament 22. As depicted in FIGS. 2A-2B, the upper end 52 of the posterior fibrous connective tissue portion 42 and/or the anterior fibrous connective tissue portion 44 can be attached to the anterior longitudinal ligament 22.

The posterior fibrous connective tissue portion 42 can be positioned within a space between the rectum 18 and the posterior vaginal wall 32, and the anterior fibrous connective tissue portion 44 can be positioned within a space between the bladder 12 and the anterior vaginal wall 34. In the embodiment of FIGS. 2A-2B, each of the posterior fibrous connective tissue portion 42 and the anterior fibrous connective tissue portion 44 may include a tibialis tendon sample having a region of increased width relative to an adjacent portion of the tendon. The region of increased width can appear to fan out from the adjacent portion in some cases. In some embodiments, the region of increased width is located at a lower end of the respective fibrous connective tissue portion and is configured for attachment to pelvic tissue. In some embodiments, this wider end can represent the lower part of the posterior fibrous connective tissue portion 42 that is sutured to the posterior vaginal wall 32. Additionally, the anterior fibrous connective tissue portion 44 can include a tibialis tendon sample having an end that fans out to a greater width as compared with the rest of the tibialis tendon sample. In some embodiments, this wider end can represent the lower part of the anterior fibrous connective tissue portion 44 that is sutured to the anterior vaginal wall 34.

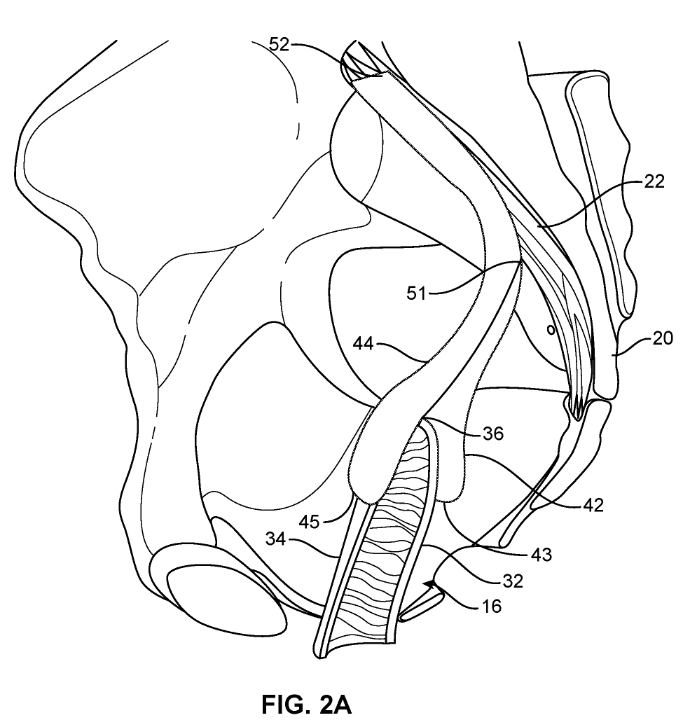

Referring now to FIG. 3, a minimally invasive pelvic procedure can include forming multiple small incisions 54A–54E in an abdomen of patient 4 to permit introduction of visualization devices and surgical instruments. One or more of the incisions 54 can receive an imaging device, and other incisions 54 can receive tools used to perform dissection, placement, and fixation of the one or more fibrous connective tissue samples 6. In some embodiments, one or more of the tools are controlled robotically. In an example where the clinician performs a laparoscopic sacrocolpopexy procedure, a clinician can make incisions 54 in an abdomen of patient 4, the incisions 54 having a diameter that is wide enough to receive long, narrow surgical tools (e.g., trocars, laparoscopes, suturing tools) but not wide enough to allow the clinician to view interior anatomy of patient 4 without imaging tools. Thus, the clinician can insert one or more imaging devices (e.g., laparoscopes or other cameras) into one or more of the incisions 54 to provide visualization during the procedure. Other ones of the incisions 54 can be used to receive other tools for performing the laparoscopic sacrocolpopexy procedure. Some or all of these tools can be tools that are controlled robotically.

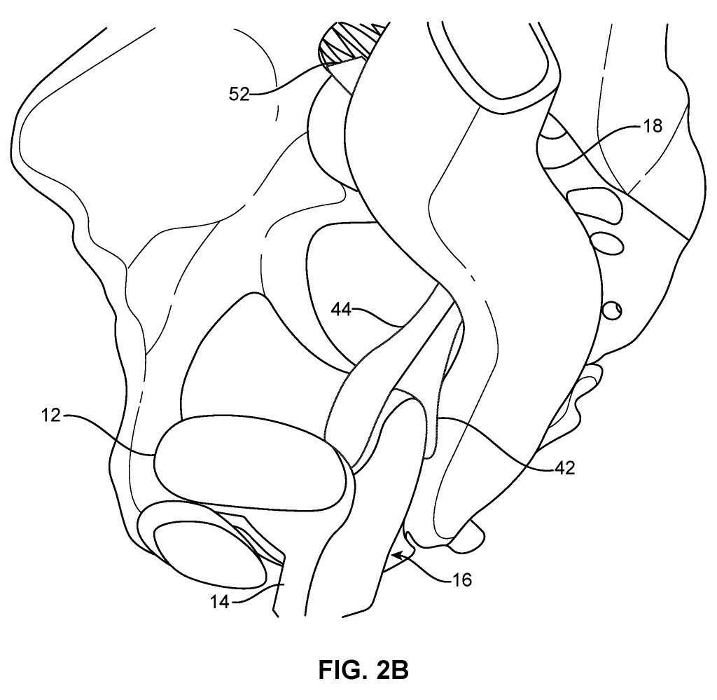

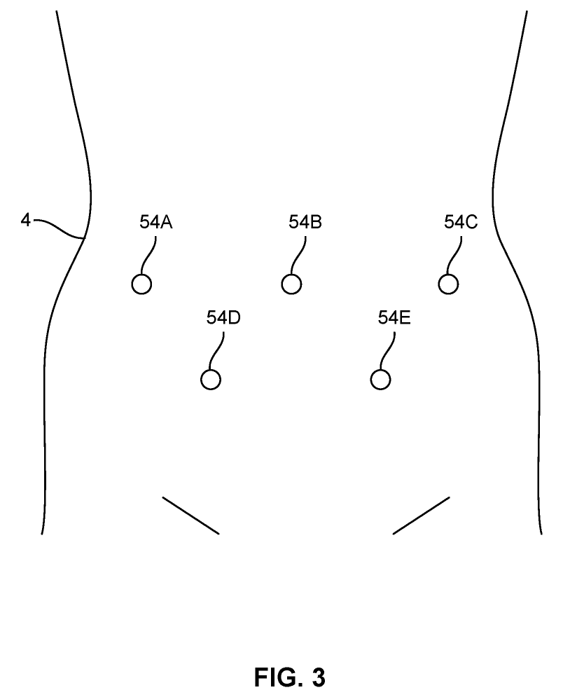

Referring now to FIGS. 4-5, the one or more fibrous connective tissue samples 6 can include a tibialis tendon sample 60. In some examples, the tibialis tendon sample 60 can extend from a lower end 62 to an upper end 64. In some embodiments, the tibialis tendon sample 60 is an example of posterior fibrous connective tissue portion 42 or anterior fibrous connective tissue portion 44 in cases where posterior fibrous connective tissue portion 42 or anterior fibrous connective tissue portion 44 are two separate tissue samples. That is, a clinician can use two samples that are each similar to tibialis tendon sample 60. In such cases, both of the tibialis tendon samples can be sutured to the walls of the vaginal vault 36 and the anterior longitudinal ligament 22 over the sacrum 20. For example, a lower part 66 of the tibialis tendon sample 60 can be sutured to an upper part of the vaginal vault 36 (e.g., an upper part of the posterior vaginal wall 32 when the tibialis tendon sample 60 represents the posterior fibrous connective tissue portion 42 or an upper part of the anterior vaginal wall 34 when the tibialis tendon sample 60 represents the anterior fibrous connective tissue portion 44). An upper part 68 of the tibialis tendon sample 60 can be sutured to the anterior longitudinal ligament 22 over the sacrum 20.

The tibialis tendon sample 60 can, in some examples, have a first width (W1) at the lower end 62 and a second width (W2) at the upper end 64, W1 being greater than W2 The upper end 64 of the tibialis tendon sample 60 can be narrower than the lower end 62 of the tibialis tendon sample 60, because a shape the tibialis tendon has different functional demands at the muscle and bone attachments. Where the tibialis tendon attaches to muscle, the tendon must connect to a wide area of contracting muscle fibers, so the tendon can be broader and flatter to anchor effectively. Where the tibialis tendon attaches to bone, the tendon bundles into a narrower, denser cord to fit through confined spaces around the ankle and transmit force efficiently. As a result, the upper end 64 of the tibialis tendon sample 60 can be narrower than the lower end 62 of the tibialis tendon because the upper end 64 attaches to muscle and the lower end 62 attaches to bone. The tibialis tendon can flare at upper part 68 to create a greater attachment area to distribute force, but overall, the bone-anchored end of the tibialis tendon can be narrower than the muscle-anchored end of the tibialis tendon. In some cases, the lower part 66 of the tibialis tendon sample 60 can be flared to distribute force over a larger area.

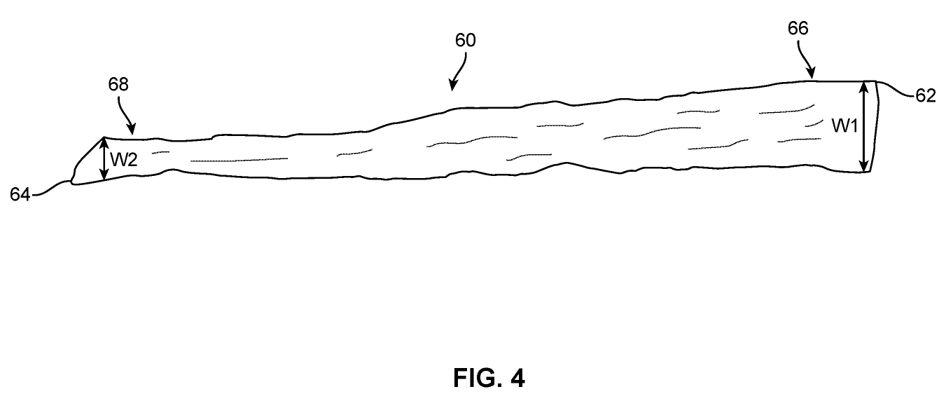

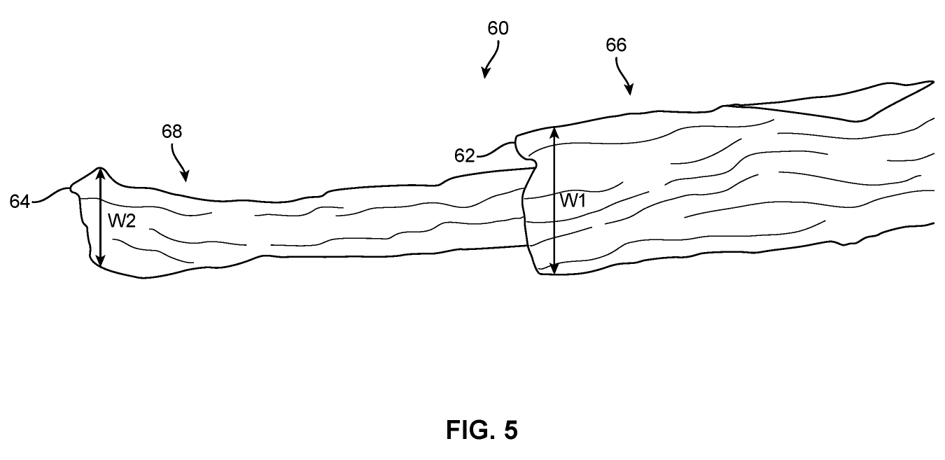

Referring now to FIGS. 6-8, the one or more fibrous connective tissue samples 6 can include a tibialis tendon sample 90. In some embodiments, the tibialis tendon sample 90 includes a first flared end portion 92 and a second flared end portion 94. The first flared end portion 92 and the second flared end portion 94 can each have a width greater than the width of adjacent portions of the tendon. These flared regions can provide an expanded surface area for attachment to soft tissue, which can facilitate securement of the tendon during use in a support construct. In some embodiments, a width of the tibialis tendon sample 90 can increase moving from a first position (P1) at an inner boundary of the first flared end portion 92 to a second position (P2) at an outer boundary of the first flared end portion 92. The width of the tibialis tendon sample 90 can also increase moving from a third position (P3) at an inner boundary of the second flared end portion 94 to a fourth position (P4) at an outer boundary of the second flared end portion 94.

In some embodiments, a greatest width of the first flared end portion 92 and a greatest width of the second flared end portion 94 can be approximately the same, but this is not required. In some embodiments, a greatest width of the first flared end portion 92 is less than a greatest width of the second flared end portion 94. In some embodiments, a greatest width of the first flared end portion 92 is greater than a greatest width of the second flared end portion 94. The first flared end portion 92 and the second flared end portion 94 can have the same length or different lengths, depending on the donor of tibialis tendon sample 90.

In some embodiments, tibialis tendon sample 90 includes flared end portions 92, 94 that define broader attachment regions than adjacent portions of the tendon. These broader attachment regions can provide increased surface area for fixation and can facilitate distribution of loads across a larger area of tissue. In some cases, the flared end portions 92, 94 flare out at the ends of tibialis tendon sample 90 because of how tendons connect to multiple bony insertions. Tendons can include bundles of collagen fibers that transfer force generated by a muscle to bone. When a tendon attaches to more than one site, the tendon can fan out to distribute the force from the muscle over a larger area. For example, the tibialis tendon increases in diameter as it approaches the medial cuneiform and the base of the first metatarsal, and the tibialis posterior tendon spreads into several slips that attach to multiple tarsal bones. This flaring creates a broader area of attachment, which reduces stress concentration at a single point.

In some embodiments, the flared end portions 92, 94 can enhance mechanical performance of tibialis tendon sample 90 by providing broader fixation regions and facilitating distribution of loads at tissue attachment sites. A tendon that broadens before insertion can provide better leverage and more effective transmission of muscular force across a joint. For tibialis tendons, flaring is important because these tendons control the arch of the foot and fine-tuning of ankle motion. The tibialis posterior tendon, for example, supports the medial longitudinal arch, and a wide insertion of the tibialis posterior tendon can help maintain balance between different bones of the midfoot. Without flaring, tibialis tendon could be more prone to tearing.

In some embodiments, the flared end portions 92, 94 can be configured such that one flared end portion is attached to the posterior vaginal wall 32 and the other flared end portion is attached to the anterior vaginal wall 34. The flared end portions 92, 94 can provide increased surface area for fixation at these attachment sites and can facilitate distribution of loads across a broader area of tissue. Because the tibialis tendon includes two flared ends (e.g., flared end portions 92, 94), tibialis tendon sample 90 is beneficial for using as a fibrous connective tissue samples for performing a sacrocolpopexy. This is because one of the first flared end portion 92 and the second flared end portion 94 can be attached to the posterior vaginal wall 32 and the other one of the first flared end portion 92 and the second flared end portion 94 can be attached to the anterior vaginal wall 34 to secure the vaginal vault 36 in place. Just as the flared end portions 92, 94 distribute force over a larger area when the tibialis tendon is still attached to the cadaveric donor, the flared end portions 92, 94 distribute force over a larger area when used as part of a tissue graft for performing a sacrocolpopexy. For example, flared end portions 92, 94 can be attached to a larger surface area of the vaginal wall, thus distributing force more effectively as compared with tissue samples that do not have flared ends.

In some embodiments, tibialis tendon sample 90, which represents a sample of one tibialis tendon from a single cadaveric donor, can be used to create a graft for performing a sacrocolpopexy procedure to secure the vaginal vault 36 to the sacrum 20. In some embodiments, a length of the tibialis tendon sample 90 (L1) can be within a range from 15cm to 40cm. In some embodiments, L1 can be within a range from 20cm to 30cm, or within a range from 25 cm to 30cm. In some embodiments, L1 can be approximately 28cm. In any case, the tibialis tendon sample 90 can have a length L1 that is suitable for crafting the tibialis tendon sample 90 into a graft that attaches to the posterior vaginal wall 32, the anterior vaginal wall 34, and a ligamentous structure proximate to the sacrum 20.

In some examples, to create the graft for performing a sacrocolpopexy procedure, a clinician can cut the tibialis tendon sample 90 at a cut location 96 located between the first flared end portion 92 and the second flared end portion 94. This can result in the tibialis tendon sample 90 being separated into a first tibialis tendon portion 98 having a length (L2) and a second tibialis tendon portion 100 having a length (L3). In some examples, the length L2 is within a range from 5cm to 12cm. In some examples, the length L2 is 8cm. In some examples, the length L3 is within a range from 10cm to 25cm, such as 20cm. As depicted in FIGS. 6-8, the length L2 is less than the length L3, meaning that the first tibialis tendon portion 98 is shorter than the second tibialis tendon portion 100. The first tibialis tendon portion 98 can extend between a first end 102 and a second end 104. The second tibialis tendon portion 100 can extend between a first end 106 and a second end 108.

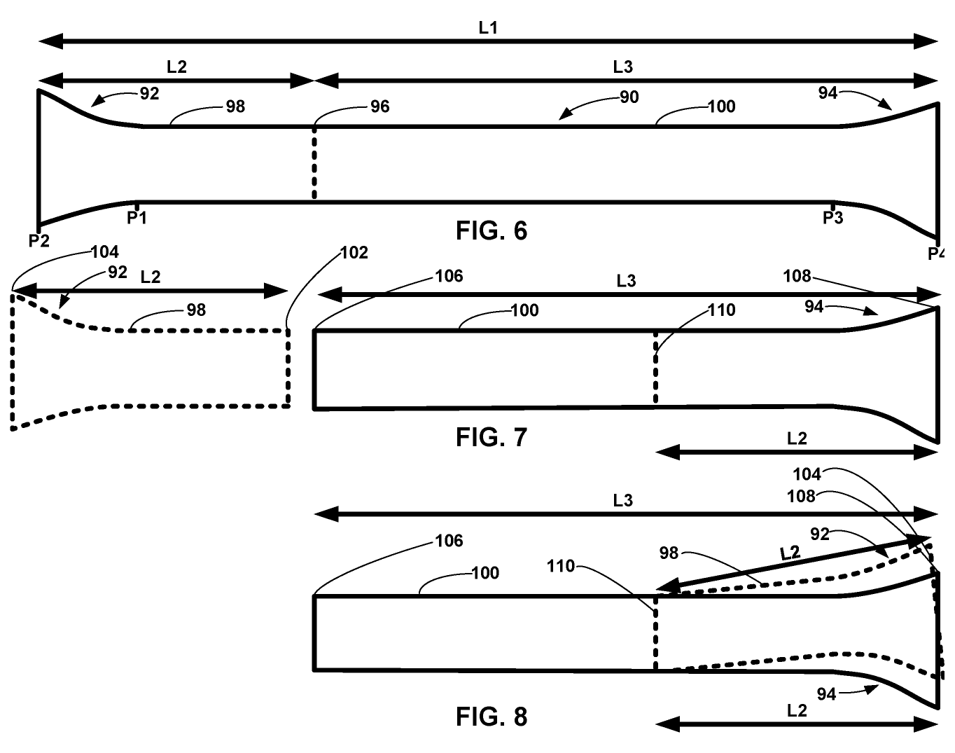

In some embodiments, the first tibialis tendon portion 98 can be attached to the second tibialis tendon portion 100 at an attachment location 110 such that the first tibialis tendon portion 98 extends from the second tibialis tendon portion 100. This configuration can position both the first flared end portion 92 and the second flared end portion 94 at a common end of the graft (depicted in FIG. 8). The flared end portions 92, 94 can then be attached to the posterior vaginal wall 32 and the anterior vaginal wall 34, respectively. In some embodiments, the first end 102 of the first tibialis tendon portion 98 can be attached to an attachment location 110 of the second tibialis tendon portion 100 such that the first tibialis tendon portion 98 extends from the attachment location 110 of the second tibialis tendon portion 100 towards the second end 108 of the second tibialis tendon portion 100. One of the first flared end portion 92 and the second flared end portion 94 can be attached to the posterior vaginal wall 32 and the other one of the first flared end portion 92 and the second flared end portion 94 can be attached to the anterior vaginal wall 34. In some examples, the graft depicted in FIG. 8 represents an example of the one or more fibrous connective tissue samples 6 depicted in FIG. 1D.

In some embodiments, the first tibialis tendon portion 98 can be attached to the second tibialis tendon portion 100 using one or more fixation techniques selected to maintain the relative positions of the tendon portions during use. Suitable fixation techniques can include sutures or other attachment mechanisms. For example, first tibialis tendon portion 98 can be connected to the second tibialis tendon portion 100 using one or more 2-0 polytetrafluoroethylene (PTFE) uninterrupted sutures in some embodiments. For example, 2-0 PTFE uninterrupted sutures represent continuous stitches made from PTFE, a synthetic, non-absorbable material known for being smooth, biocompatible, and resistant to infection. Unlike interrupted sutures, where each stitch is tied off individually, an uninterrupted (or continuous) suture can use a single strand that runs along the attachment site without breaks, distributing tension evenly across the tissue. PTFE’s soft, monofilament-like structure minimizes tissue drag and inflammation, making it beneficial for attaching tendons to create grafts. In some examples, the sutures have spacing of between 5 millimeters (mm) and 7mm and can be 3-4mm from an edge of the graft.

The graft that results from attaching first tibialis tendon portion 98 to the second tibialis tendon portion 100 can define a “Y” shape including a stem portion extending between the first end 106 of the second tibialis tendon portion 100 and the attachment location 110, and two branch portions extending from the attachment location 110. In some cases, a distance between the attachment location 110 and the second end 108 of the second tibialis tendon portion 100 is approximately the same length (L2) as the length of the first tibialis tendon portion 98 between the first end 102 and the second end 104. This means that the second end 104 of the first tibialis tendon portion 98 can occupy approximately the same axial position as the second end 108 of the second tibialis tendon portion 100 when the first end 102 of the first tibialis tendon portion 98 is attached to the attachment location 110.

In some embodiments, tibialis tendon sample 90 can be divided and reconfigured such that both flared end portions are positioned at a common end of a graft, with a remaining portion configured for fixation to a ligamentous structure proximate to the sacrum 20. The resulting configuration can define a multi-point support construct. In some implementations, the tibialis tendon sample 90 is 28cm in length including a natural flare at each end (e.g., flared end portions 92, 94). The mid portion of the tibialis tendon sample 90 can be tapered (e.g., the ends can be wider). As described above, the tibialis tendon sample 90 can be cut 8cm from one of the flared ends (e.g., first flared end portion 92) and transposed to the opposite end of tibialis tendon sample 90 so that both arms of the “Y” shape include a flared end. In the final configuration of the graft created using the tibialis tendon sample 90, the second tibialis tendon portion 100 can represent a posterior portion of the graft. The first end 106 of the second tibialis tendon portion 100 can represent a blunt, non-flared cut. In some cases, a clinician can attach the first end 106 of the second tibialis tendon portion 100 to the sacrum 20 and the clinician can attach one of the first flared end portion 92 and the second flared end portion 94 to the posterior vaginal wall 32 and attach the other one of the first flared end portion 92 and the second flared end portion 94 to the anterior vaginal wall 34.

In some embodiments, the Y-shaped configuration of tibialis tendon sample 90, as depicted in FIG. 8, can provide a mechanically robust support construct capable of maintaining anatomical positioning under physiological loading conditions. In some embodiments, the Y-shaped graft depicted in FIG. 8 can withstand more than 100 Newtons (N) of tension applied to the graft without sutures dislodging from the anterior longitudinal ligament 22 over the sacrum 20, the posterior vaginal wall 32, or the anterior vaginal wall 34, and without the second tibialis tendon portion 100 dislodging from the first tibialis tendon portion 98. The tibialis tendon can be beneficial for use in a graft for a sacrocolpopexy procedure, because the tibialis tendon can exhibit a tensile strength within a range from 60 MegaPascals (MPa) to 100 MPa. This tensile strength is far in excess of the load placed on the graft during daily activity.

In some embodiments, prior to implant, the graft can be preserved using one or more preservation techniques. Suitable preservation techniques can include freezing, freeze-drying (lyophilization), or storage in a preservation solution. The graft can be stored and transported under conditions selected to maintain the structural integrity of the tissue. In some embodiments, prior to implant, the graft can be stored in a frozen environment (e.g., in -40 degrees Celsius). In some embodiments, the graft can be stored in a Glycerol-based solution at room temperature. The graft can be transported in a frozen environment or another storage environment that enables preservation.

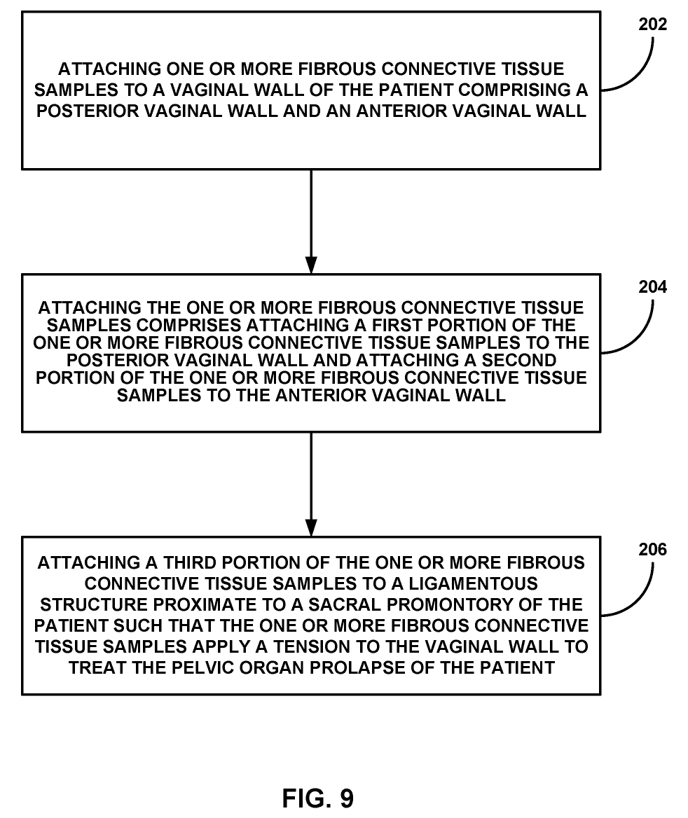

FIG. 9 is a flow diagram illustrating an example technique for providing structural support to pelvic anatomy using one or more fibrous connective tissue samples 6. FIG. 9 is described with respect to system 2 of FIGS. 1A-1D. However, the techniques of FIG. 9 can be performed by different components of system 2 or by additional or alternative systems.

For example, a method includes attaching one or more fibrous connective tissue samples 6 to a vaginal wall of the patient 4 comprising a posterior vaginal wall 32 and an anterior vaginal wall 34 (202). In some cases, attaching the one or more fibrous connective tissue samples 6 to the vaginal wall of the patient 4 comprises attaching a first portion of the one or more fibrous connective tissue samples 6 to the posterior vaginal wall 32 and attaching a second portion of the one or more fibrous connective tissue samples 6 to the anterior vaginal wall 34 (204). The first portion of the one or more fibrous connective tissue samples 6 can include, for example, the posterior fibrous connective tissue portion 42 and the second portion of the one or more fibrous connective tissue samples 6 can include the anterior fibrous connective tissue portion 44. In some embodiments, the method includes attaching third portion of the one or more fibrous connective tissue samples 6 to a ligamentous structure proximate to a sacral promontory of the patient 4 such that the one or more fibrous connective tissue samples 6 apply a tension to the vaginal wall to treat the pelvic organ prolapse of the patient 4 (206).

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method for performing a surgical procedure to treat a pelvic organ prolapse of a patient using a biologic graft, the method comprising:
attaching one or more fibrous connective tissue samples to a vaginal wall of the patient comprising a posterior vaginal wall and an anterior vaginal wall, wherein attaching the one or more fibrous connective tissue samples comprises:
attaching a first portion of the one or more fibrous connective tissue samples to the posterior vaginal wall; and
attaching a second portion of the one or more fibrous connective tissue samples to the anterior vaginal wall; and
attaching a third portion of the one or more fibrous connective tissue samples to a ligamentous structure proximate to a sacral promontory of the patient such that the one or more fibrous connective tissue samples apply a tension to the vaginal wall to treat the pelvic organ prolapse of the patient.

2. The method of claim 1, wherein:
the one or more fibrous connective tissue samples comprise a first fibrous connective tissue sample and a second fibrous connective tissue sample;
attaching the first portion comprises attaching a first end of the first fibrous connective tissue sample to the posterior vaginal wall; and
attaching the second portion comprises attaching a first end of the second fibrous connective tissue sample to the anterior vaginal wall.

3. The method of claim 2, wherein:
attaching the one or more fibrous connective tissue samples to the ligamentous structure comprises:
attaching a second end of the first fibrous connective tissue sample to the ligamentous structure, a second end of the second fibrous connective tissue sample being attached to the first fibrous connective tissue sample at a junction located between the first end of the first fibrous connective tissue sample and the second end of the first fibrous connective tissue sample,
wherein the one or more fibrous connective tissue samples comprise one or more tibialis tendon samples.

4. The method of claim 2, wherein:
attaching the one or more fibrous connective tissue samples to the ligamentous structure comprises:
attaching a second end of the second fibrous connective tissue sample to the ligamentous structure, a second end of the first fibrous connective tissue sample being attached to the second fibrous connective tissue sample at a junction located between the first end of the second fibrous connective tissue sample and the second end of the second fibrous connective tissue sample,
wherein the one or more fibrous connective tissue samples comprise one or more tibialis tendon samples.

5. The method of claim 2, wherein:
attaching the one or more fibrous connective tissue samples to the ligamentous structure comprises attaching a second end of the first fibrous connective tissue sample and a second end of the second fibrous connective tissue sample to the ligamentous structure,
wherein the one or more fibrous connective tissue samples comprise one or more tibialis tendon samples.

6. The method of claim 1, wherein one or both of the first portion and the second portion comprises a first section having a first width and a second section having a second width, the second width being greater than the first width; and
wherein:
attaching the first portion of the one or more fibrous connective tissue samples comprises attaching the second section of the first fibrous connective tissue sample to the posterior vaginal wall; or
attaching the second portion of the one or more fibrous connective tissue samples comprises attaching a first end of the second fibrous connective tissue sample to the anterior vaginal wall.

7. The method of claim 1, wherein:
the one or more fibrous connective tissue samples comprise a single fibrous connective tissue sample,
attaching the single fibrous connective tissue sample to the vaginal wall comprises:
attaching a first end of the single fibrous connective tissue sample to the posterior vaginal wall, and
attaching a second end of the single fibrous connective tissue sample to the anterior vaginal wall; and
attaching the single fibrous connective tissue sample to the ligamentous structure comprises attaching a portion of the single fibrous connective tissue sample between the first end and the second end to the ligamentous structure.

8. The method of claim 1, wherein the one or more fibrous connective tissue samples comprise one or more tibialis tendon samples.

9. The method of claim 8, wherein the one or more tibialis tendon samples comprise at least one of a posterior tibialis tendon sample or an anterior tibialis tendon sample.

10. The method of claim 1, wherein each of the one or more fibrous connective tissue samples defines a maximum length, a maximum width, and a maximum depth, the maximum length being greater than the maximum width and the maximum depth, and the maximum width being greater than the maximum depth.

11. The method of claim 10, wherein the maximum width is about 4 centimeters.

12. The method of claim 10, wherein the maximum width is no less than about 4 centimeters.

13. The method of claim 1, wherein attaching the one or more fibrous connective tissue samples to the vaginal wall of the patient comprises attaching the one or more fibrous connective tissue samples to the vaginal wall using absorbable fixation material.

14. The method of claim 13, wherein the absorbable fixation material comprises absorbable sutures.

15. The method of claim 1, wherein attaching the one or more fibrous connective tissue samples to the ligamentous structure proximate to the sacral promontory comprises attaching the one or more fibrous connective tissue samples to the ligamentous structure using non-absorbable fixation material.

16. The method of claim 1, wherein the method further comprises covering at least a portion of the one or more fibrous connective tissue samples with peritoneum.

17. The method of claim 1, wherein the method further comprises, prior to attaching the one or more fibrous connective tissue samples to the vaginal wall and the ligamentous structure proximate to the sacral promontory:
decellularizing the one or more fibrous connective tissue samples to remove one or more cellular components while preserving an extracellular matrix; and
sterilizing the one or more fibrous connective tissue samples.

18. The method of claim 1, wherein each fibrous connective tissue sample of the one or more fibrous connective tissue samples defines a length from 15 centimeters to 25 centimeters.

19. The method of claim 18, wherein the length is about 21 centimeters.

20. The method of claim 1, wherein:
attaching the one or more fibrous connective tissue samples to the vaginal wall of the patient comprises:
using a robotic system to attach the one or more fibrous connective tissue samples to the vaginal wall of the patient; and
attaching the third portion of the one or more fibrous connective tissue samples to the ligamentous structure proximate to the sacral promontory of the patient comprises
using the robotic system to attach third portion of the one or more fibrous connective tissue samples the ligamentous structure proximate to the sacral promontory of the patient.

21. The method of claim 1, wherein the one or more fibrous connective tissue samples comprise a tibialis tendon, and wherein both ends of the tendon are fanned out to create broad suture platforms, with one end sutured to the anterior vaginal wall, the other end sutured to the posterior vaginal wall, and a midpoint of the tendon fixed to the ligamentous structure proximate to the sacral promontory, thereby forming a Y-shaped biologic suspension graft as a biologic alternative to synthetic mesh.
